(19) United States
(12) Patent Application Publication
(10) Pub. No.: US 2022/0338912 A1
(43) Pub. Date: Oct. 27, 2022

(54) BONE LOCKING SYSTEM AND METHOD

(71) Applicant: JBone Biotechnology Co., Ltd., Taoyuan City (TW)

(72) Inventor: CHINE-MING LEE, TAOYUAN CITY (TW)

(21) Appl. No.: 17/235,948

(22) Filed: Apr. 21, 2021

(51) Int. Cl. A61B 17/86; A61B 17/80; A61B 17/70

(52) U.S. Cl. CPC A61B 17/8665; A61B 17/8014; A61B 17/7059; A61B 2017/564

(57) ABSTRACT

A bone locking system is applied to join multiple broken bone fragments, including a self-tapping nut having a cylindrical structure, the self-tapping nut having an outside surface including an external threading portion, the external threading portion being formed, through recessing, a tapping notch, the self-tapping nut having an inside surface including an internal threading portion, a tool portion being formed in the inside surface of the self-tapping nut at an upper end thereof, the self-tapping nut being embedded in a cortical bone portion of each broken bone fragment; and a fixing bolt corresponding to the internal threading portion of the self-tapping nut, the fixing bolt being screwed into and fixed in the self-tapping nut to join each of broken bone fragments. A bone plate having a fixing hole is selectively provided. The fixing bolt penetrates through the fixing hole of the bone plate and is screwed into the self-tapping nut.

### TECHNICAL FIELD OF THE INVENTION

The present invention relates to a system and a method for fixing a broken bone, and more particularly to a bone locking system and method that increase locking strength and guiding effect.

### DESCRIPTION OF THE PRIOR ART

When a bone is damaged and broken due to wound, disease, or various sorts of surgery, in order to allow the facture bone to steadily heal back to the original configuration, a bone plate and fixing screws are used to fixed broken pieces of the fracture bone. Since the bone marrow is a loose part, the known bone screws A shown in FIG. 1 must penetrate through the bone in order to allow the two opposite ends of the fixing screw A to be fixed to the cortical bone part of the hard outer layer of the bone to securely fix all broken pieces of the fracture bone, but is incapable of twisting in order to prevent the bone from collapsing. Consequently, the known fixing screw A would be set in contact with the bone with a substantial length and area. Once the bone heals, the known fixing screw A has to be removed. The known fixing screw A is susceptible to combinative coupling with the bone plate, or the known fixing screw A may have a thread breaking or thread shifting situation. This makes the removal of the known fixing screw A and the bone plate difficult, or the removal of the known fixing screw A and the bone plate may cause secondary damages. Or, when the patient suffers osteoporosis or the cortical bone is thin, a situation as shown in FIG. 2 that the known fixing screws A detach or separate from the broken bone may happen.

### SUMMARY OF THE INVENTION

The primary objective of the present invention is to provide a bone fixing system and method, in which a combination of self-tapping nut and bolt is used to replace the known fixing screw, in order to increase the fastening strength and guiding effect of a bone nail and to allow the bolt and bone plate to be removed in an effortless manner and also reducing secondary damage.

The bone locking system according to the present invention is applicable to join a plurality of broken bone fragments and comprises at least one self-tapping nut, the self-tapping nut being of a cylindrical structure, the self-tapping nut having an outside surface that includes an external threading portion, the external threading portion being formed, through recessing, with at least one tapping notch, the self-tapping nut having an inside surface that includes an internal threading portion, the inside surface of the self-tapping nut being provided with a tool portion, the self-tapping nut being positionable, as being embedded, in a cortical bone portion of each of the broken bone fragments, the tapping notch receiving bone chips generated by tapping in screwing the self-tapping nut into each of the broken bone fragments so as to have the self-tapping nut securely embedded in each of the broken bone fragments; and at least one fixing bolt, which corresponds to the internal threading portion of the self-tapping nut, the fixing bolt being screwed into and fixed in the self-tapping nut to join each of the broken bone fragments, wherein the self-tapping nut forms an internally-threaded hole, which is of an increased strength, in each of the broken bone fragments to receive each of the fixing bolts to screw therein and fixed thereto, so that there is no need for each of the fixing bolts to penetrate through each of the broken bone fragments.

The bone locking system according to the present invention may additionally include, if desired, a bone plate. The bone plate is formed with a plurality of fixing holes. Each of the fixing bolts penetrates each of the fixing holes and is screwed into and fixed to each of the self-tapping nuts to apply a force to tightly clamp the bone plate to join each of the broken bone fragments.

The bone locking method according to the present invention comprises the following steps: hole drilling, in which a guide device is used in forming, through drilling, at least one mounting cavity in each of the broken bone fragments, each mounting cavity being not penetrating through each of the broken bone fragments; screwing self-tapping nut, in which a self-tapping nut is screwed into and fixed in the mounting cavity, the self-tapping nut being embedded in a cortical bone portion of each of the broken bone fragments, the self-tapping nut being of a cylindrical structure, the self-tapping nut having an outside surface that includes an external threading portion, the external threading portion being a tapping screw thread, the external threading portion being formed, through recessing, with at least one tapping notch, the self-tapping nut having an inside surface that includes an internal threading portion, the inside surface of the self-tapping nut being provided with a tool portion at an upper end thereof, the tool portion being provided for a tool to rotate the self-tapping nut; and screwing and fixing, in which at least one fixing bolt is screwed into and fixed in the self-tapping nut fixed in the mounting cavity to join each of the broken bone fragments, the fixing bolt corresponding to each internal threading portion of the self-tapping nut.

The bone locking method according to the present invention may additionally include, if desired, a step of disposing bone plate, the step of disposing bone plate being after the step of screwing self-tapping nut and before the step of screwing and fixing, the step of disposing bone plate comprising disposing at least one bone plate on each of the broken bone fragments, the bone plate being formed with a plurality of fixing holes, each mounting cavity corresponding, in position, to each of the fixing holes; the step of screwing and fixing further comprising penetrating a plurality of fixing bolts through each fixing hole for being screwed into and fixed in each of self-tapping nuts located in each mounting cavity to apply a force to tightly clamp the bone plate to joint each of the broken bone fragments, each fixing bolt corresponding to each internal threading portion of each self-tapping nut.

The present invention enables easy screwing of each fixing bolt, by means of first implanting a self-tapping nut and then disposing, if desired, a bone plate, to be tightly abutting the bone plate, and also allows easy operation of removal of the bone plate and each fixing bolt. This would overcome the problem of sticking of a prior art fixing bolt to the fixing hole of the bone plate in mounting the prior art fixing bolt to the bone plate, or thread breaking or thread shifting occurring in the prior art fixing bolt, which lead to the situation that the prior art fixing bolt and the bone plate are difficult to remove after the broken bone fragment heals.

The self-tapping nut forms an internally-threaded hole, which is of an increased strength, in each broken bone fragment to receive the fixing bolt to screw therein and fixed thereto, so that there is no need for the fixing bolt to penetrate through each broken bone fragment, thereby reducing the extent of combinative coupling between the fixing bolt and the bone plate due to tissue proliferation. The fixing bolt and the bone plate can be removable more easily after the healing of the broken bone fragments. It is also possible to prevent the occurrence of detachment or separation of the fixing bolt due to the patient suffering osteoporosis or the cortical bone being thin, and the depth of hole drilling can be reduced and the healing of each broken bone fragment can be accelerated. If the self-tapping nut is made of a biologically non-toxic material and is long kept in each broken bone fragment, re-adjustment of each broken bone fragment can be applied in a later date, if desired. Each tapping notch provided in the outside surface of the self-tapping nut is applicable to receive bone chips generated by a tapping operation applied for screwing into each broken bone fragment in order to have the self-tapping nut more securely embedded in each broken bone fragment. Using a guide device in hole drilling may help ensure the angular position of hole drilling does not deviate from a predetermined angle.

The present invention is applicable to all portion of a human body, including the spine, and is also applicable to bone fracture caused by all sorts of wound and damage.

The tool portion can be a star-shaped hole with threading. The tool portion is provided for a star-shaped tool to rotate and screw the self-tapping nut into the mounting cavity. The tapping notch can be rectangular in shape and is formed, through recessing, in a lower end of the external threading portion.

### BRIEF DESCRIPTION OF THE DRAWINGS

FIG. 1 is a schematic view showing a known way of fixing a broken bone.

FIG. 2 is another schematic view showing a known way of fixing a broken bone.

FIG. 3 is a perspective view showing a bone locking system according to a first embodiment of the present invention.

FIG. 4 is a flow chart showing a bone locking method according to the first embodiment of the present invention.

FIG. 5 is a first schematic view demonstrating an operation of the bone locking method according to the first embodiment of the present invention.

FIG. 6 is a second schematic view demonstrating an operation of the bone locking method according to the first embodiment of the present invention.

FIG. 7 is a third schematic view demonstrating an operation of the bone locking method according to the first embodiment of the present invention.

FIG. 8 is a fourth schematic view demonstrating an operation of the bone locking method according to the first embodiment of the present invention.

FIG. 9 is a schematic view demonstrating a bone locking method according to a second embodiment of the present invention.

FIG. 10 is a schematic view demonstrating a bone locking method according to a third embodiment of the present invention. FIG. 11 is a schematic view demonstrating a bone locking method according to a fourth embodiment of the present invention.

FIG. 12 is a schematic view demonstrating a bone locking method according to a fifth embodiment of the present invention.

FIG. 13 is a schematic view demonstrating a bone locking method according to a sixth embodiment of the present invention.

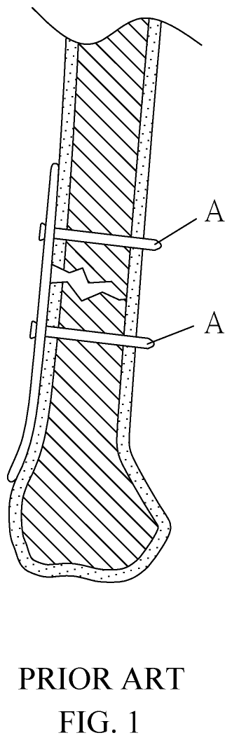

### DETAILED DESCRIPTION OF THE PREFERRED EMBODIMENT

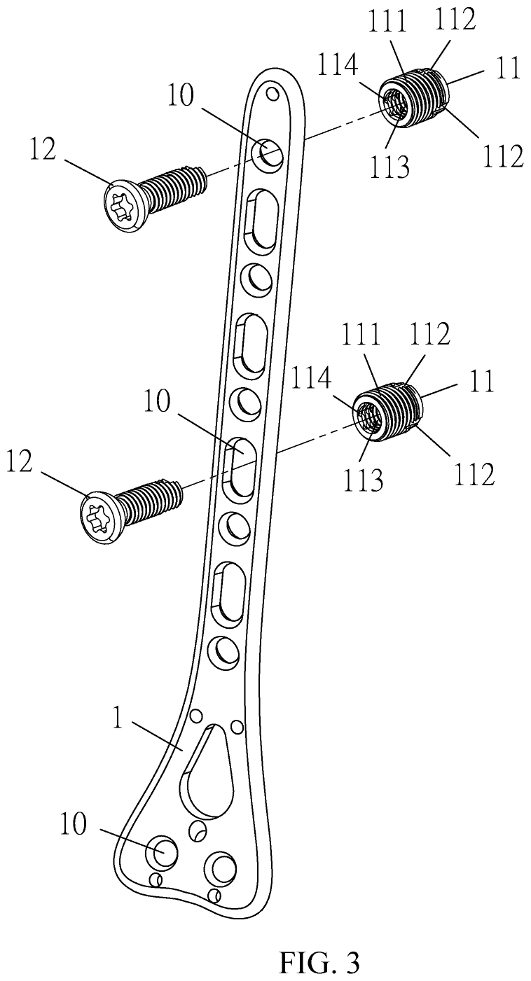

Referring to FIG. 3, a drawing showing an embodiment of the present invention is provided. A bone locking system according to a first embodiment of the present invention, which is applicable to join a plurality of broken bone fragments B, comprises at least one self-tapping nut 11. The self-tapping nut 11 is of a cylindrical structure. The self-tapping nut 11 has an outside surface that includes an external threading portion 111. The external threading portion 111 can be a tapping screw thread. The external threading portion 111 is formed, through recessing, with at least one tapping notch 112. In the instant embodiment, the external threading portion 111 is recessed and formed with three rectangular tapping notches 112 in a lower end thereof. Each tapping notch 112 in the outside surface of the self-tapping nut 11 is applicable to receive bone chips generated by a tapping operation applied to screw the self-tapping nut 11 into each broken bone fragment B, in order to have the self-tapping nut 11 more securely embedded in each broken bone fragment B. In the instant first embodiment, each tapping notch 112 is a rectangular notch, but the present invention does not impose any limitation to the shape of each tapping notch 112. The self-tapping nut 11 has an inside surface that includes an internal threading portion 113. The inside surface of the self-tapping nut 11 is provided with a tool portion 114 at an upper end thereof. The self-tapping nut 11 is embedded in each broken bone fragment B in such a way that the tool portion 114 orients outwards. The self-tapping nut 11 is screwed into and fixed in a cortical bone portion of each broken bone fragment B. In the instant embodiment, the tool portion 114 is arranged in the form of a star-shaped hole with threading. The tool portion 114 is provided for a star-shaped tool to rotate the self-tapping nut 11 to drive and embed the self-tapping nut 11 into and in the cortical bone portion of the broken bone fragment B. The threading of the tool portion 114 is identical to the internal threading portion 113. At least one fixing bolt 12 corresponds to each internal threading portion 113 of the self-tapping nut 11. The fixing bolt 12 is screwed and fixed to the self-tapping nut 11 to join each broken bone fragment B. The self-tapping nut 11 forms an internally-threaded hole, which is of an increased strength, in each broken bone fragment B to receive the fixing bolt 12 to screw therein and fixed thereto, so that there is no need for the fixing bolt 12 to penetrate through each broken bone fragment B, and so that detachment or separation of the fixing bolt 12 does not readily occur even though the patient suffers osteoporosis or the cortical bone is thin.

The bone locking system according to the first embodiment of the present invention further comprises a bone plate 1, which is formed with a plurality of fixing holes 10. The plurality of fixing holes 10 allows the bone plate 1 of the present invention to correspond to the broken bone fragment B in any fracture position. The present invention does not impose any limitation to the shape of the bone plate 1 and the distribution and number of the fixing holes 10. The shape of the bone plate 1 and the distribution and number of the fixing holes 10 can be modified as desired according to the shape of the bone at the site of application. For example, for applications to such as the spine, the palm, or the leg, the bone plate 1 may be made in different shapes. In the use of the bone plate 1, mounting cavity 2, the self-tapping nut 11, and the fixing bolt 12 may be provided in multiplicity to securely fix the bone plate 1. Each fixing bolt 12 penetrates each fixing hole 10 and then screw and fix to each self-tapping nut 11 and applies a force to tightly clamp the bone plate 1 to join each broken bone fragment B. Each broken bone fragment B can be one of the spinal bones, so that the present invention is applicable to spine surgery, such as spinal fusion surgery and is not limited to treatment of bone fracture. The mounting cavity 2 can be formed as being drilled in a front side, a lateral side, or a back side of each spinal bone.

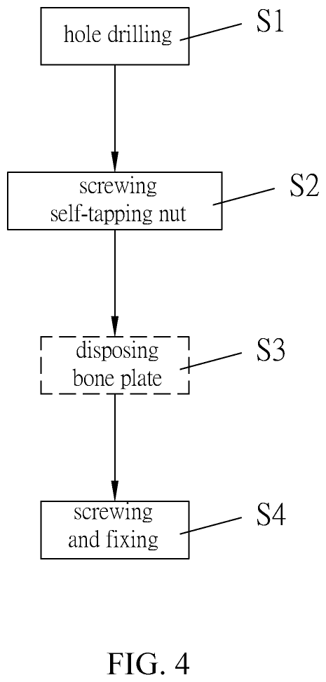

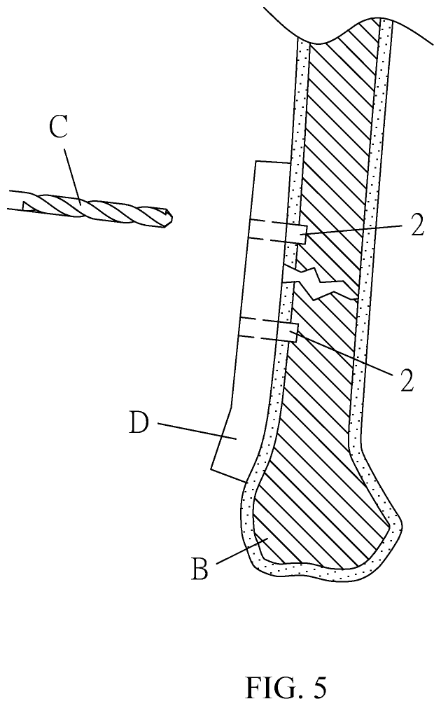

As shown in FIG. 4, a bone locking method according to the present invention is applied to join a plurality of broken bone fragments B. Steps of the bone locking method comprises: hole drilling S1, in which a guide device D is used in forming, through drilling, at least one mounting cavity 2 in each broken bone fragment B in such a way that the mounting cavity 2 does not penetrate through each broken bone fragment B; screwing self-tapping nut S2, in which a self-tapping nut 11 is screwed into and fixed in the mounting cavity 2 in such a way that the self-tapping nut 11 is screwed into and fixed in the cortical bone portion of each broken bone fragment B, wherein the self-tapping nut 11 is of a cylindrical structure; the self-tapping nut 11 has an outside surface that includes an external threading portion 111; the external threading portion 111 is a tapping screw thread; the external threading portion 11 is formed, through recessing, with at least one tapping notch 112; the self-tapping nut 11 has an inside surface that includes an internal threading portion 113; the inside surface of the self-tapping nut 11 is provided with a tool portion 114 in an upper end thereof; the tool portion 114 is provided for a tool to rotate and screw the self-tapping nut 11; and the tapping notch 112 is applicable to receive bone chips generated by a tapping operation applied to screw the self-tapping nut 11 into each broken bone fragment B, in order to have the self-tapping nut 11 more securely embedded in each broken bone fragment B; and screwing and fixing S4, in which at least one fixing bolt 12 is screwed into and fixed to the self-tapping nut 11 located in the mounting cavity 2 to join each broken bone fragment B, wherein the fixing bolt 12 corresponds to the internal threading portion 113 of the self-tapping nut 11.

The bone locking method according to the first embodiment of the present invention further comprises a step of disposing bone plate S3, in which after the step of screwing self-tapping nut S2 and before the step of screwing and fixing S4, the step of disposing bone plate S3 comprises disposing at least one bone plate 1 on each broken bone fragment B, wherein the bone plate 1 is formed with a plurality of fixing holes 10, the numbers of the mounting cavity 2 and the self-tapping nut 11 being in multiplicity, and each mounting cavity 2 is set to correspond, in position, to each fixing hole 10; and the step of screwing and fixing S4 further comprises penetrating a plurality of fixing bolts 12 through each fixing hole 10 for being screwed into and fixed in each self-tapping nut 11 located in each mounting cavity 2 and applying a force to tightly clamp the bone plate 1 to join each broken bone fragment B, wherein each fixing bolt 12 corresponds to each internal threading portion of each self-tapping nut 11. For surgery requiring no the bone plate 1, the step of disposing bone plate S3 can be omitted, wherein a block drawn with phantom lines in FIG. 3 indicates the step of disposing bone plate S3 can be selectively omitted.

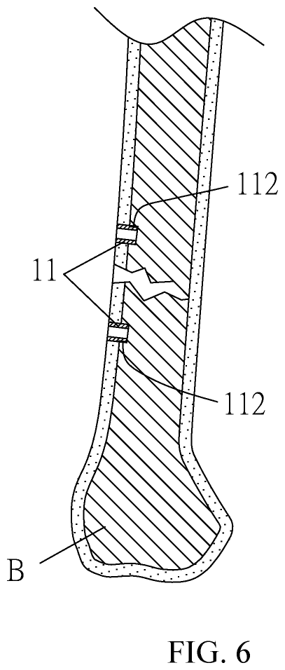

In FIG. 5, it can be seen in the step of hole drilling Si according to the present invention, a drill bit C and the guide device D are used to drill and thus form at least one mounting cavity 2 in each broken bone fragment B, wherein using the guide device D to assist performing the step of hole drilling Si may help ensure the mounting cavity 2 that is so formed through drilling is set at a correct angular position. In the instant embodiment, two mounting cavities 2 are involved, and it can be seen that each of the mounting cavities 2 does not penetrate each broken bone fragment B.

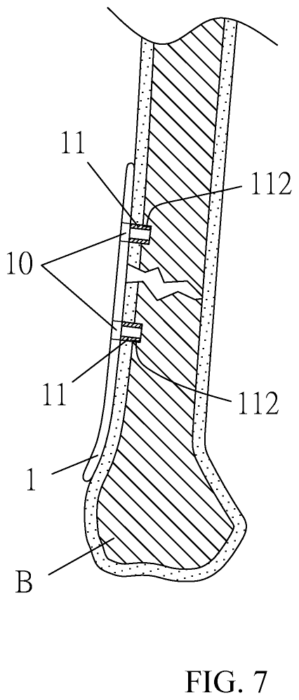

In FIG. 6, it can be seen in the step of screwing self-tapping nut Si according to the present invention, a self-tapping nut 11 is driven and screwed into each mounting cavity 2, and each self-tapping nut 11 is screwed into and fixed in the cortical bone portion of each broken bone fragment B, wherein as noted above, each self-tapping nut 11 is of a cylindrical structure; each self-tapping nut 11 has an outside surface that includes an external threading portion 111; each external threading portion 111 is formed, through recessing, with at least one tapping notch 112, and in the instant embodiment, each tapping notch 112 has a rectangular shape and is formed, through recessing, in a lower end of each external threading portion 111, the number of which is three; each self-tapping nut 11 is embedded in each broken bone fragment B in a direction that each tool portion 114 orients outwards so as to have each tapping notch 112 embedded in each broken bone fragment B; each self-tapping nut 11 has an inside surface that includes an internal threading portion 113; each self-tapping nut 11 is provided, in the inside surface thereof, with a tool portion 114 at an upper end thereof; each tool portion 114 is provided to allow a tool to rotate each self-tapping nut 11, wherein in the instant embodiment, each tool portion 114 is arranged in the form of a star-shaped hole with threading; the threading of each tool portion 114 is identical to each internal threading portion 113; each tool portion 114 is provided for a star-shaped tool to rotate each self-tapping nut 11 to driven and screw each self-tapping nut 11 into the broken bone fragment B; each tapping notch 112 provided in the outside surface of each self-tapping nut 11 is applicable to receive bone chips generated by a tapping operation applied for screwing into each broken bone fragment B in order to have each self-tapping nut 11 more securely embedded in each broken bone fragment B; each self-tapping nut 11 can be made of a biologically non-toxic material and can be long kept in each broken bone fragment B for application of re-adjustment of each broken bone fragment B in a later date.

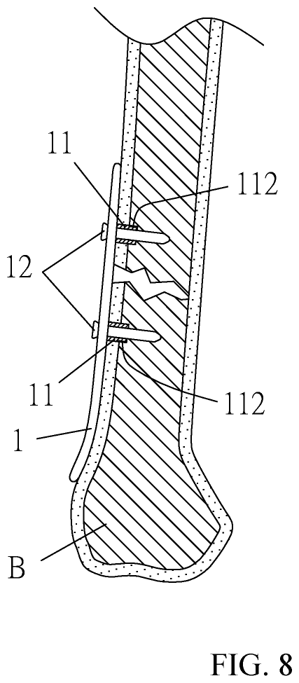

In FIG. 7, it can be seen in the step of disposing bone plate S3 according to the present invention, at least one bone plate 1 is disposed on each broken bone fragment B, wherein the bone plate 1 is formed with a plurality of fixing holes 10, and each mounting cavity 2 is set to correspond, in position, to each fixing hole 10, wherein the present invention does not impose any limitation to the shape of the bone plate 1 and the distribution and number of the fixing holes 10, and the shape of the bone plate 1 and the distribution and number of the fixing holes 10 can be modified as desired according to the shape of the bone at the site of application, and for example, for applications to such as the spine, the palm, or the leg, the bone plate 1 may be made in different shapes, and in the use of the bone plate 1, the self-tapping nut 11 and the fixing bolt 12 may be provided in multiplicity to securely fix the bone plate 1, and each fixing bolt 12 penetrates each fixing hole 10 and then screw and fix to each self-tapping nut 11 and applies a force to tightly clamp the bone plate 1 to join each broken bone fragment B.

In FIG. 8, it can be seen in the step of screwing and fixing S4 according to the present invention, a plurality of fixing bolts 12 penetrate each fixing hole 10 and is then screwed into and fixed in each self-tapping nut 11 located in each mounting cavity 2 to apply a force to tightly clamp the bone plate 1 so that the bone plate 1 joins each broken bone fragment B, wherein each fixing bolt 12 corresponds each internal threading portion 113 of each self-tapping nut 11.

Each self-tapping nut 11 forms an internally-threaded hole, which is of an increased strength, in each broken bone fragment B to receive each fixing bolt 12 to screw therein and fixed thereto, so that there is no need for each fixing bolt 12 to penetrate through each broken bone fragment B. In this way, mutual combinative coupling between each fixing bolt 12 and the bone plate 1 can be reduced and removal of the fixing bolt 12 and the bone plate 1 is easy after the broken bone fragment B heals. Since there is no need to drilling through each broken bone fragment B, the depth of hole drilling is reduced and fast healing of each broken bone fragment B can be achieved.

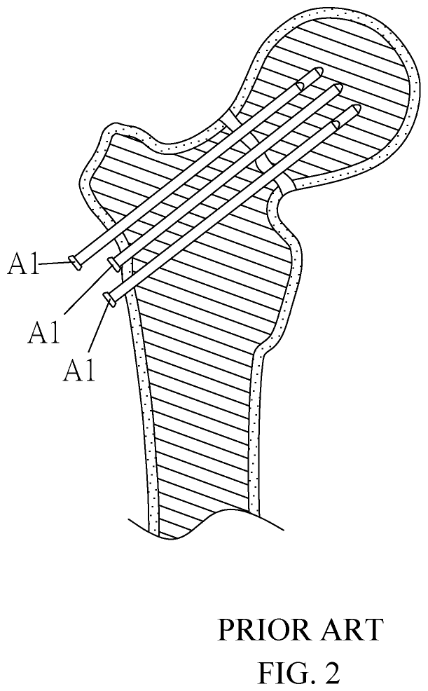

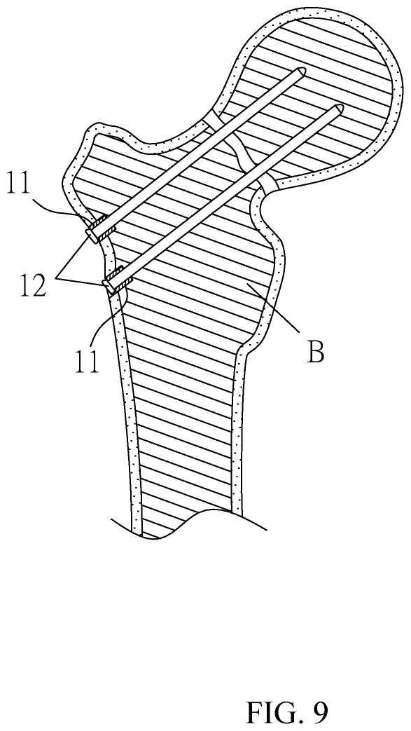

In FIG. 9, in a bone locking method according to a second embodiment of the present invention, a therapeutic operation where no bone plate 1 is shown, and only a plurality of fixing bolts 12 and a plurality of self-tapping nuts 11 are involved. The second embodiment of the present invention is applied to fix the site where a femoral bone is connected to a hip joint. The present invention helps avoid the situation of FIG. 2 where detachment and separation of the fixing bolt Al occurs.

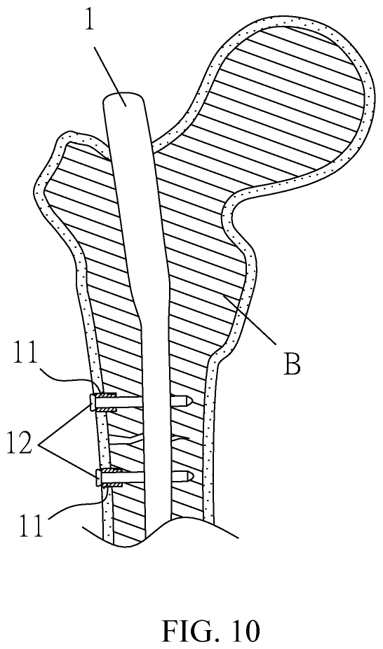

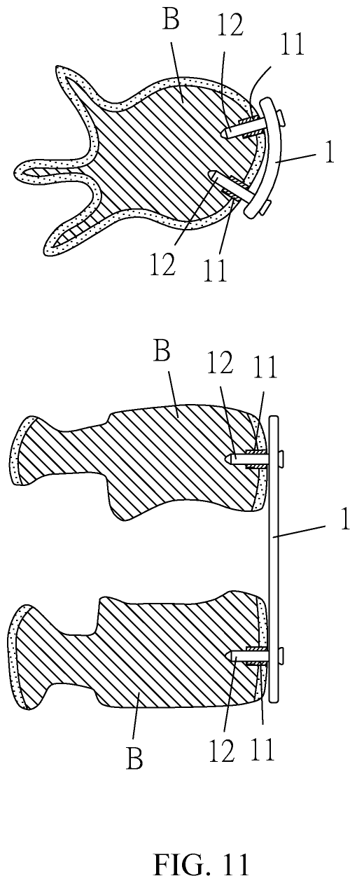

In FIG. 10, in a bone locking method according to a third embodiment of the present invention, it can be seen that the present invention may securely fix the bone plate 1, and each fixing bolt 12 is securely screwed into and fixed in each self-tapping nut 11. The present invention helps prevent the occurrence of detachment and separation of each fixing bolt 12.

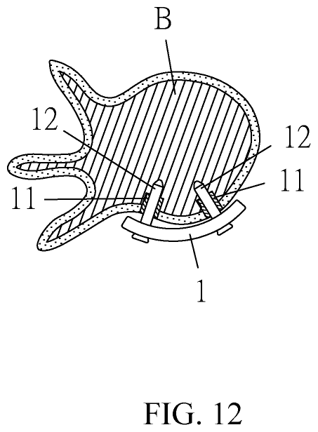

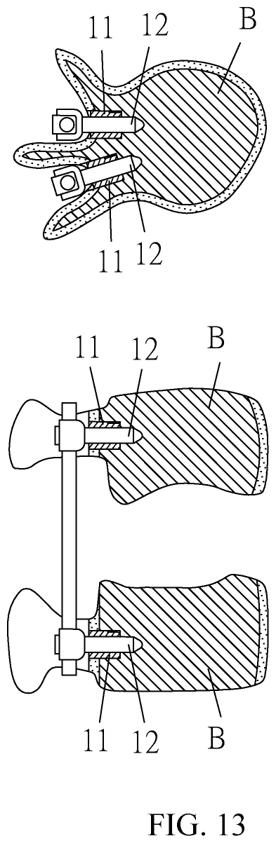

In FIGS. 11-13, in bone locking method according to fourth, fifth, and sixth embodiments of the present invention, each broken bone fragment B can be a spinal bone, and the present invention is applicable to spine surgery, such as spinal fusion surgery, and is not limited to treatment of bone fracture. The mounting cavity 2 and the self-tapping nut 11 can be embedded in a front side, a lateral side, or a back side of each spinal bone according to the requirement of the surgery, and may be selectively used in combination with other devices, such as the bone plate 1, a bone nail (not shown), or a nail bar (not shown).

The present invention enables easy screwing of each fixing bolt, by means of first implanting a self-tapping nut 11 and disposing, if desired, a bone plate 1, to be tightly abutting the bone plate 1, and also allows easy operation of removal of the bone plate 1 and each fixing bolt 12. This would overcome the problem of sticking of a prior art fixing bolt A to the fixing hole 10 of the bone plate 1 in mounting the prior art fixing bolt A to the bone plate 1, or thread breaking or thread shifting occurring in the prior art fixing bolt A, which lead to the situation that the prior art fixing bolt A and the bone plate 1 are difficult to remove after the broken bone fragment B heals. The self-tapping nut 11 also provides an effect of guiding the fixing bolt 12, and could help prevent detachment or separation of each fixing bolt 12 from each broken bone fragment B due to a patient suffering osteoporosis or the cortical bone being thin.

1. A bone locking system, which is adapted to join a plurality of broken bone fragments, comprising:
at least one self-tapping nut, the self-tapping nut being of a cylindrical structure, the self-tapping nut having an outside surface that includes an external threading portion, at least one tapping notch being formed in the external threading portion-being, the self-tapping nut having an inside surface that includes an internal threading portion, the inside surface of the self-tapping nut being provided with a tool portion, the self-tapping nut being positionable, as being embedded, in a cortical bone portion of each of the broken bone fragments, the tapping notch receiving bone chips generated by tapping in screwing the self-tapping nut into each of the broken bone fragments so as to have the self-tapping nut securely embedded in each of the broken bone fragments; and
at least one fixing bolt, which corresponds to the internal threading portion of the self-tapping nut, the fixing bolt being screwed into and fixed in the self-tapping nut to join each of the broken bone fragments,
wherein the tool portion of the self-tapping nut comprises threading formed in the inside surface of the self-tapping nut and extending to an end of the self-tapping nut, the tool portion comprising internal notches formed in the threading such that the notches define a hole of the tool portion that has a predetermined shape, wherein the threading of the tool portion of the self-tapping nut is identical to the internal threading portion of the self-tapping nut.

2. The bone locking system according to claim 1, wherein the tool portion is formed as a star-shaped hole, and the tool portion is provided for a star-shaped tool to rotate the self-tapping nut.

3. The bone locking system according to claim 1, further comprising a bone plate, the bone plate being formed with a plurality of fixing holes, wherein the fixing bolts penetrate each of the fixing holes and are screwed into and fixed to the self-tapping nut to apply a force to tightly clamp the bone plate to join each of the broken bone fragments.

4. The bone locking system according to claim 1, wherein each of the broken bone fragments comprises a spinal bone.

5. A bone locking method, which is applicable to join a plurality of broken bone fragments, the bone locking method comprising the following steps:
hole drilling, in which a guide device is used in forming, through drilling, at least one mounting cavity in each of the broken bone fragments, each mounting cavity being not penetrating through each of the broken bone fragments;
self-tapping nut screwing, in which a self-tapping nut is screwed into and fixed in each mounting cavity, the self-tapping nut being embedded in a cortical bone portion of each broken bone fragment, the self-tapping nut being of a cylindrical structure, the self-tapping nut having an outside surface that includes an external threading portion, at least one tapping notch being formed in the external threading portion, the self-tapping nut having an inside surface that includes an internal threading portion, the inside surface of the self-tapping nut being provided with a tool portion at an upper end thereof, the tool portion being provided for a tool to rotate the self-tapping nut, the tapping notch receiving bone chips generated by tapping in screwing the self-tapping nut into each of the broken bone fragments so as to have the self-tapping nut securely embedded in each of the broken bone fragments, wherein the tool portion of the self-tapping nut comprises threading formed in the inside surface of the self-tapping nut and extending to the upper end of the self-tapping nut, the tool portion comprising internal notches formed in the threading such that the notches define a hole of the tool portion that has a predetermined shape corresponding to a shape of the tool, wherein the threading of the tool portion of the self-tapping nut is identical to the internal threading portion of the self-tapping nut; and
bolt screwing and fixing, in which at least one fixing bolt is screwed into and fixed in the self-tapping nut fixed in the mounting cavity to join each of the broken bone fragments, the fixing bolt corresponding to the internal threading portion of the self-tapping nut.

6. The bone locking method according to claim 5, further comprising a step of bone plate disposing, wherein the step of bone plate disposing is performed after the step of screwing self-tapping nut screwing and before the step of bolt screwing and fixing, the step of bone plate disposing comprising disposing at least one bone plate on each of the broken bone fragments, the bone plate being formed with a plurality of fixing holes, numbers of the mounting cavity and the self-tapping nut being in multiplicity, each mounting cavity corresponding, in position, to each of the fixing holes; the step of bolt screwing and fixing further comprising penetrating a fixing bolt of a plurality of fixing bolts through each fixing hole for being screwed into and fixed in each of the self-tapping nuts located in each mounting cavity to apply a force to tightly clamp the bone plate to joint each of the broken bone fragments, each fixing bolt corresponding to each internal threading portion of each self-tapping nut.

7. The bone locking system according to claim 5, wherein each of the broken bone fragments comprises a spinal bone.
